(19) United States
(12) Patent Application Publication
(10) Pub. No.: US 2015/0011824 A1
(43) Pub. Date: Jan. 8, 2015

(54) Endoscope with Electrically Adjustable Liquid Crystal Adaptive Lens

(71) Applicant: Yi Sun Wilkinson, San Francisco, CA (US)

(72) Inventor: Yi Sun Wilkinson, San Francisco, CA (US)

(21) Appl. No.: 14/325,151

(22) Filed: Jul. 7, 2014

Related U.S. Application Data
(60) Provisional application No. 61/843,828, filed on Jul. 8, 2013.

(51) Int. Cl. A61B 1/00; A61B 1/05

(52) U.S. Cl. CPC A61B 1/0019; A61B 1/05
USPC 600/109

(57) ABSTRACT

Various embodiments of an endoscope capable of varying a focal length electrically are disclosed. In one embodiment, the endoscope comprises an optical imaging system within an inner portion of the elongate tube, wherein the optical imaging system comprises a liquid crystal adaptive lens (LCAL) comprising a ground plate, a first reference plate, a first liquid crystal layer and a first plurality of closed-loop electrodes configured to receive variable control voltages and a control system configured to adjust variable control voltages. In another embodiment, the LCAL in the endoscope may further comprise a second reference plate, a second liquid crystal layer and a second plurality of closed-loop electrodes.

### BACKGROUND OF THE INVENTION

The present invention relates generally to endoscope systems, and in particular, an endoscope comprising a liquid crystal adaptive lens capable of varying a focal length electrically.

In endoscopy and related fields, rigid or flexible elongate narrow tubes are used to observe an internal structure within a human body through a natural opening or a small incision for clinical inspection and treatment. A conventional endoscope comprises an illumination system, an optical imaging system, and a viewing system (an eye piece or image sensor). An endoscope has a distal end and a proximate end. The images of an endoscope have better quality when using video chip technology on the distal end instead of on the proximate end.

A conventional endoscope usually has a fixed focal length and a wide field of view to observe an overall internal structure and find an area of interest. However, the magnification of the image of such a system is too low to provide enough information for diagnosis and treatment. It is difficult to achieve a variable focal length in an endoscope. It is more difficult to achieve a variable focal length endoscope with high magnification and large focal length adjustable range that is able to withstand the sterilization procedure in the autoclave.

### SUMMARY OF THE INVENTION

The present invention provides an endoscope capable of varying a focal length electrically by using a liquid crystal adaptive lens (LCAL). The endoscope comprises an optical imaging system within an inner portion of the elongate tube, wherein the optical imaging system comprises an LCAL. In one embodiment, the LCAL comprises a ground plate, a first reference plate, a first liquid crystal layer and a first plurality of closed-loop electrodes configured to receive variable control voltages and a control system configured to adjust variable control voltages. In order to increase light transmission, in another embodiment, the LCAL in the endoscope may further comprise a second reference plate, a second liquid crystal layer and a second plurality of closed-loop electrodes wherein the second liquid crystal layer is aligned in a direction perpendicular to that of the first liquid crystal layer. The endoscope may comprise a distance sensing subsystem, an auto-focusing subsystem and an aberration correction subsystem. The focal length of the endoscope may be adjusted in milliseconds with a large focal length varying range. The endoscope is further capable of displaying a three-dimensional image. The endoscope is suitable to withstand the sterilization procedure in the autoclave at 140° C. for about one hour.

In one embodiment, an endoscope comprises an elongate tube, a transparent window, an illumination system, an optical imaging system comprising an LCAL, an image sensor and a control system. The illumination system comprises solid state emitters or fiber bundles located at an outer portion of the elongate tube. The LCAL comprises a ground plate, a first reference plate, a first liquid crystal layer and a first plurality of closed loop electrodes disposed on the first reference plate, configured to receive variable control voltages. The image sensor is configured to receive an image from the optical system and converts optical signals to electrical signals. The control system receives electrical signals from the image sensor, processes the signals and adjusts variable control voltages of the LCAL, thus changing the focal length of the endoscope. Because the response time of the liquid crystal molecules is in milliseconds, the focal length of the endoscope can be adjusted with a speed in the order of kHz.

In another embodiment, an endoscope comprises a double cell LCAL wherein the LCAL comprises a first reference plate, a first liquid crystal layer, and a first plurality of closed-loop electrodes as well as a second reference plate, a second liquid crystal layer, and a second plurality of closed-loop. The second liquid crystal layer is aligned in a direction perpendicular to that of the first liquid crystal layer. The double cell LCAL allows light polarized in all directions to pass through the optical imaging system. Because of the limited available light sources and limited space, sufficient light transmission is an important factor in endoscopic application. The double cell LCAL provides the advantage of double increased light transmission rate. In order to minimize the aberration resulting from the distance between the two liquid crystal layers, both liquid crystal layers share the same ground plate which may be a super thin transparent substrate.

In yet another embodiment, the endoscope comprises an LCAL emulating a Fresnel phase profile. The closed-loop electrodes in the LCAL comprise at least one subset of closed-loop electrodes comprising a Fresnel zone. To provide variable control voltages to the closed-loop electrodes, the LCAL further comprises at least one pair of conductors connected with at least two closed-loop electrodes, and at least one connector electrically connecting at least two closed-loop electrodes and each conductor of a respective pair of conductors. The LCAL with a Fresnel phase profile reduces the overall aberration of the optical imaging system.

In one embodiment, the endoscope comprises an auto-focusing subsystem in the control system. The control system applies a set of control voltages to the closed-loop electrodes. The image sensor receives an image of an internal structure formed by the optical imaging system. The image sensor converts optical signals to electrical signals. The control system receives electrical signals from the image sensor and calculates the point spread function of the received image. Then the control system increases the voltages and repeats the process. As such, the control system may compare the point spread function for the received image at different voltages and determine the optimum control voltages for the endoscope system.

In another embodiment, the control system of an endoscope further comprises an aberration correction subsystem. In order to minimize both dynamic and static aberrations, the aberration correction subsystem calculates an aberration evaluation function by analyzing the image from the optical system. The aberration evaluation function may be defined by using several methods. For example, “knife edging technique” may be used to analyze the light intensity at the edges of the image to evaluate the overall aberration. The subsystem determines the control voltages corresponding to the minimum aberration evaluation function.

In yet another embodiment, an endoscope comprises a distance-sensing subsystem. The distance-sensing subsystem is located at the distal end. It comprises a LED, optically couple to a collimating lens such that the LED delivers collimated light. It further comprises a beam splitter, which is used to direct a portion of the reflected light from an internal structure to a photodiode. The photodiode converts the optical signals to electrical signals. The control system receives the electrical signals from the photodiode and calculates the distance of the internal structure to the LED. The control system further comprises a look-up table which maps the desired control voltages with the calculated distance. The distance sensing subsystem greatly facilitates the process of determining the desired control voltages. Therefore, the endoscope with a distance-sensing subsystem may adjust to the desired control voltages in sub-milliseconds.

In another embodiment, the endoscope is configured to use wireless communication. A battery is located at the distal end of the elongate tube to provide electricity to the illumination system, the LCAL, the image sensor and the control system. A wireless transmitter is connected to the image sensor to receive signals from the image sensor and broadcasts the signals to a wireless transceiver at the base. A monitor displays the image from the signals received by the wireless transceiver. The control system is also connected to the image sensor to receive signals from the image sensor. The control system may also comprise a distance-sensing subsystem, an auto-focusing subsystem and an aberration correction system.

In one embodiment, the transparent window is located on the front surface of the elongate tube. In another embodiment, the transparent window is located on the side wall. In yet another embodiment, the transparent window is aligned at an angle to the front surface. The various different configurations of the transparent window allow the physicians to have different viewing angles of the internal structure of the patient.

In another embodiment, the optical imaging system of the endoscope comprises an objective lens, a relay system, a series of rod lenses, an eye piece and an LCAL located at the proximal end near the eyepiece. This configuration allows the physicians to observe the internal structure with their eyes. It also avoids introducing electricity into the human bodies.

In one embodiment, an endoscope comprising a LCAL is capable of displaying a three-dimensional image wherein the control system further comprises an imaging process subsystem. The imaging process subsystem is configured to analyze a series of two-dimensional received images at a series of focal length, extract depth information from the two-dimensional images, and generate a three-dimensional image from the series of two-dimensional images with extracted depth information.

In another embodiment, an endoscope is capable of forming an electrically adjustable focal length three-dimensional image by comprising a second image sensor. The optical axis of the first image sensor and the optical axis of the second image sensor has a small convergent angle, which result in a three dimensional image.

### BRIEF DESCRIPTION OF THE DRAWINGS

FIG. 1 is a schematic illustration of an endoscope with an electrically variable focal length according to one embodiment of the invention.

FIG. 2 is a schematic side view of the LCAL according to one embodiment of the present invention.

FIG. 3 is a schematic top view of the LCAL according to one embodiment of the present invention.

FIG. 4 is a cross-section view of the section of the LCAL taken along line A according to one embodiment of the present invention.

FIG. 5 is a schematic side view of a double cell LCAL according to one embodiment of the present invention.

FIG. 6 is a flow diagram of the auto-focusing subsystem of the control system according to one embodiment of the present invention.

FIG. 7 is a schematic view of an endoscope comprises a distance-sensing subsystem according to one embodiment of the present invention.

FIG. 8 is a flow diagram of an aberration correction subsystem of the endoscope control system according to one embodiment of the present invention.

FIG. 9 is a schematic illustration of a wireless module of an endoscope with an electrically variable focal length according to one embodiment of the invention.

FIG. 10 is a schematic view of an endoscope with a transparent window at the side wall according to one embodiment of the present invention.

FIG. 11 is a schematic view of an endoscope with a front surface at an angle according to one embodiment of the present invention.

FIG. 12 is a schematic view of an endoscope with fiber bundles as an illumination source according to one embodiment of the present invention.

FIG. 13 is a schematic view of an endoscope with an eyepiece according to one embodiment of the present invention.

FIG. 14 is a schematic view of an endoscope capable of forming a three-dimensional image with an electrically adjustable focal length according to another embodiment of the present invention.

The figures are only for purposes of illustration only. Those skilled in the art will recognize that there are other alternative embodiments within the scope of the present invention.

### DETAILED DESCRIPTION OF THE INVENTION

The present invention now will be described in detail with reference to the accompanying figures. This invention may be embodied in many different forms and should not be construed as limited to the example embodiments discussed herein.

Embodiments of the present invention comprise endoscopes capable of electrically varying the focal length. In some embodiments, the endoscope has an elongate tube, a transparent window, an illumination system, an optical imaging system, a control system and a viewing system. The illumination system delivers light to an internal structure of a human being. The light reflected from the internal structure passes through the optical imaging system and forms an image on the viewing system. A control system adjusts the focal length of the optical imaging system to produce a high resolution and high quality image. The focal length of the endoscope can be electrically adjusted without moving parts with a fast speed in the order of kHz.

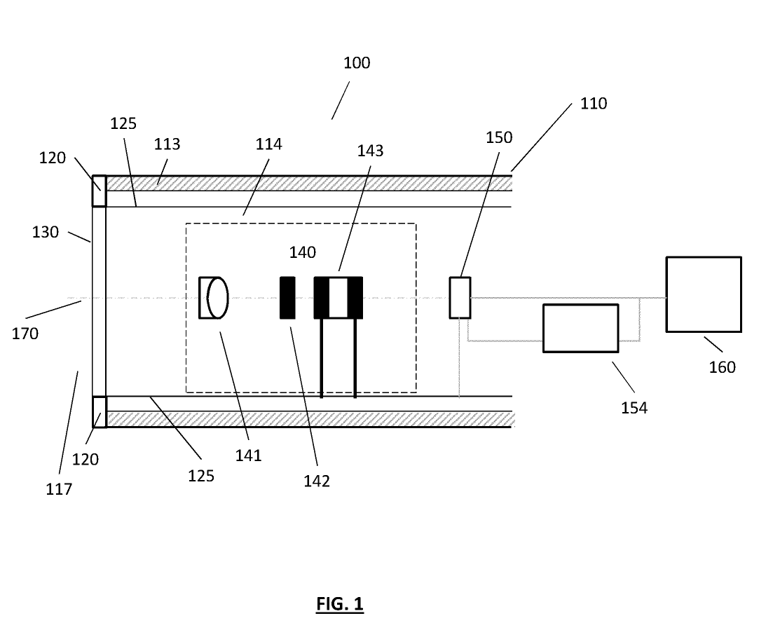

FIG. 1 is a schematic illustration of an endoscope with an electrically variable focal length according to one embodiment of the invention. An endoscope 100 capable of electrically varying a focal length comprises an elongate tube 110 wherein the elongate tube 110 has an outer portion 113 and an inner portion 114 with a distal end 117. There is a set of solid state emitters 120 located at the outer portion 113 of the elongate tube 110 with a set of wires 125 to provide electricity to the solid state emitters 120. The solid state emitters may be of a small size and high intensity. For example, the solid state emitters 120 may be light emitting diodes (LED) less than 1 millimeter in diameter. The small size the solid state emitters will provide the advantage of a less intrusive operation.

A transparent window 130 is adapted to be at a front surface at the distal end 117. The optical imaging system 140 is adapted to be within the inner portion 114 of the tube 110, wherein the optical imaging system 140 comprises an objective lens 141, a polarizer 142 and a liquid crystal adaptive lens (LCAL) 143. The objective lens 141 and the polarizer 142 are aligned with an optical axis 170 of the LCAL 143. An image sensor 150 is also aligned along the optic axis 170. The image sensor 150 has an optical input and an electrical output. It may be a CCD (charge coupled diode) device. The image sensor 150 is configured to receive an image from the optical system 140 and converts optical signals to electrical signals. A control system 154 is communicatively coupled to the image sensor 150 and receives the electrical signals from the image sensor 150. The control system 154 is configured to process the electrical signals and adjust the focal length of the LCAL. A display system such as a monitor 160 allows the physicians to observe the image.

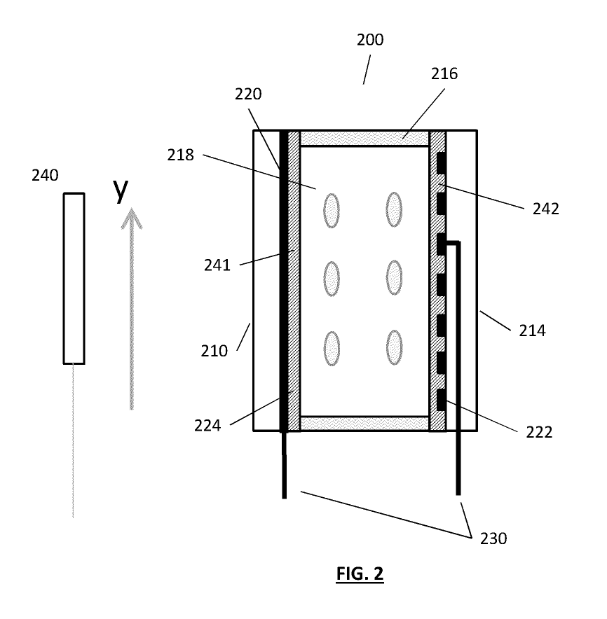

FIG. 2 is a schematic side view of the LCAL according to one embodiment of the present invention. An LCAL 200 comprises a ground plate 210, and a reference plate 214 connected to the ground plate 210 by a connecting member 216, a liquid crystal layer 218 is disposed between the ground plate 210 and the reference plate 214. A uniform conducting layer 220 is deposited on the ground plate, and a first insulating alignment layer 241 is deposited on top of the conducting layer 220. A plurality of closed-loop electrodes 222 is disposed on the reference plate. The plurality of closed-loop electrodes 222 is configured to receive a plurality of variable control voltages. A second insulating alignment layer 242 is disposed on top of the closed loop electrodes on the reference plate 214. Wires 230 provide electricity to the closed loop electrodes 222.

The ground plate 210 and the reference plate 214 may be made of any transparent materials. For example, they may be made of silicon dioxide which has a high purity and optical quality. The conducting layer 220 and the closed-loop electrodes 222 are constructed of a transparent conducting material, such as indium tin oxide (ITO). The connecting member 216 may be conventional spacers such as Myler spacer. The alignment layers 241 and 242 may be polyimide, which are specially treated by a rubbing machine to align the orientation of the liquid crystal molecules 218. After the surface of the plate is treated specially in either the “x” (not shown) or “y” direction, the liquid crystal molecules 218 will align homogeneously in either the “x” or “y” direction.

The liquid crystal layer 218 may be formed by any liquid crystal materials with a fast response time. In some embodiments, the liquid crystal layer 218 is formed by nematic liquid crystal materials with a response time of sub-milliseconds. Liquid crystal materials are electro-optical materials. The optical properties of liquid crystal, such as refractive index, may be changed electrically. However, liquid crystal materials are also polarization sensitive. If the liquid crystal molecules 218 are aligned along the “y” direction, only light polarized in that direction will be affected. Thus the LCAL 200 is configured to be used in conjunction with a polarizer 240, which is aligned in the same direction of the alignment layers 241 and 242.

The plurality of closed-loop electrodes 222 on the reference plate 214 is configured to receive a set of variable control voltages such that a refractive index of at least a portion of the liquid crystal layer 218 is adjustable. Thus light passing through the liquid crystal layer 218 is capable of being redirected, such as being brought into a focus. The refractive index across the aperture of the LCAL 200 can be graded to emulate the refractive index of a conventional lens. The focal length of the LCAL 200 is capable of being electrically adjusted by changing the set of variable control voltages. Because the liquid crystal materials have a response time less than 1 millisecond, the focal length of the LCAL 200 may be adjusted in a high speed in the order of kHz. The endoscope using an LCAL is suitable for three-dimensional imaging application because of the fast response time of the liquid crystal materials.

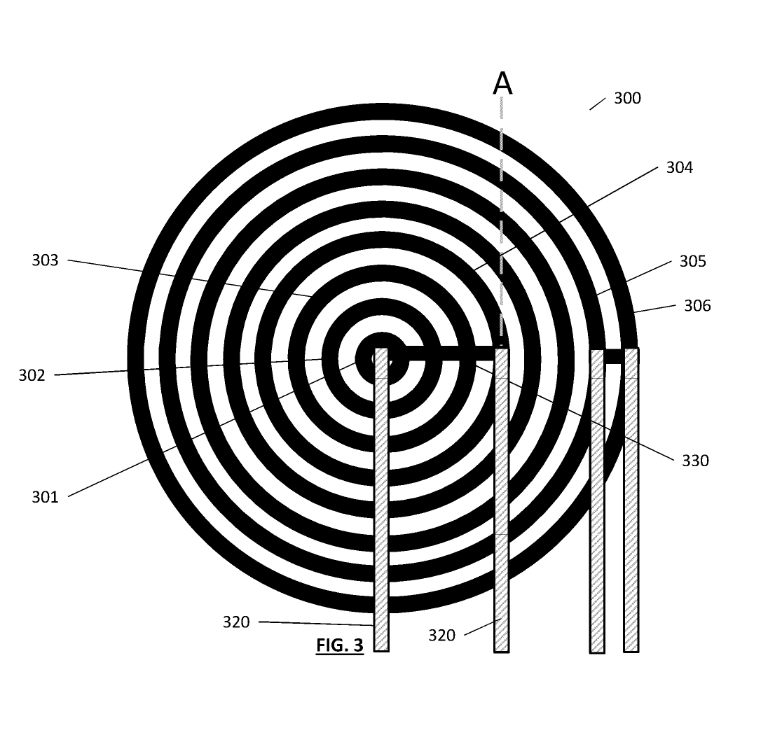

FIG. 3 is a schematic top view of the LCAL according to one embodiment of the present invention. FIG. 3 is for illustration purpose only because the electrodes are transparent and invisible to the naked eyes. A plurality of closed loop electrodes 301-306 is disposed in concentric circular pattern. In order to apply a voltage across the electrodes, the LCAL 300 comprises a set of highly conductive conductors 320 connected to respective closed-loop addressable electrodes 301 and 304 to apply the set of control voltages. The closed loop electrodes are also interconnected by a connector 330 of low conductivity to reduce the number of conductors 320. The LCAL 300 comprises at least one connector 330 electrically connecting at least two closed-loop electrodes 301-304 and at least one pair of conductors 320 in electrical contact with at least two addressable closed-loop electrodes 301 and 304.

In order to minimize aberration of LCAL 300, LCAL 300 is configured to emulate a lens with a Fresnel phase profile. A lens with a Fresnel phase profile has a set of Fresnel zones, as known to those skilled in the art. The closed-loop electrodes comprise at least one subset of electrodes 301-304, wherein each subset of closed-loop electrodes comprising a Fresnel zone. The subset of closed-loop electrodes electrically connected by a low conducting connector 330 so as to act as a Fresnel zone, with two Fresnel zones shown in FIG. 3. A pair of highly conductive conductors 320 is disposed to apply voltages to the two addressable electrodes 301 and 304 of the subset of closed loop electrodes. FIG. 3 is for illustrative purposes only because that the Fresnel zones typically comprise a greater number of electrodes and conductors.

The LCAL 300 employs an equal phase spacing design wherein the phase delay in each Fresnel zone is equal. For a nominal “design” focal length, the phase delay in each Fresnel zone is 2π. When the focal length is changed, the phase delay in each Fresnel zone will be equal, but not exactly 2π. The equal phase spacing design minimizes overall aberration, optimizes the coherent transfer function (CTF), and maximizes the variable focal length range.

The closed-loop electrodes are discrete, possibly resulting static aberration. As known to those skilled in the art, phase aberration in lenses results in blurring and loss of clearness in the images produced by the lenses. The static phase aberration in LCAL 300 includes quantization aberration and meshing aberration in addition to the conventional static aberrations. Quantization aberration is the result of sampling the refractive index of the lens by discrete electrodes, while meshing aberration results from the difference in refractive indices between the electrode region and the interstitial region (region between electrodes). The refractive index distortion in the interstitial region creates the meshing phase aberration, which causes a “lenslet” effect, thus introducing aberration. A small width of the electrodes will reduce or almost eliminate quantization aberration and meshing aberration.

The closed-loop circular electrodes are with a width of 10 nm and above and a spacing of 10 nm and above. The nano-scale feature size of the closed loop electrodes may be achieved by several techniques in nano-fabrication. Electron beam may be used to produce patterns with a feature size as small as 5 nm. Alternatively, dry etching, especially laser ablation can be used to form a pattern with a feature size of 10 nm. Dry etching technique overcomes the problem of pattern wall collapsing associated with wet etching. A sacrificial layer, such as silicon dioxide, may also be used to fabricate a pattern with a feature size of 10 nm.

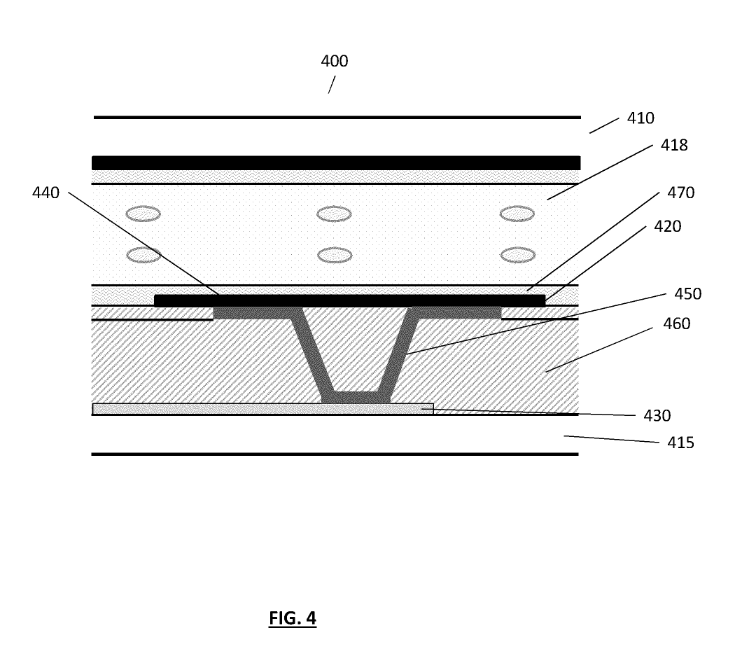

FIG. 4 is a cross-section view of the section of the LCAL taken along line A according to one embodiment of the present invention. The LCAL comprises a ground plate 410, a reference plate 415, and a liquid crystal layer 418. The LCAL 400 further comprises at least one pair of highly conductive conductors 430 to apply voltages to the electrodes 420. The circular electrodes 420 are connected by at least one connector 440 that is connected to the highly conductive conductors 430 by vias 450. The electrodes 420 and the conductors 430 are insulted by an insulating layer 460. Another insulating alignment layer 470 is disposed to separate the liquid crystal layer 418 from the electrodes 420 and the connector 440.

Because of the conductivity of the conductors 430 and connectors 440, they may be separated in the LCAL by insulating layers, including a base insulating layer 460 and a planarizing insulating layer 470. In one embodiment, the insulating layer 460 is formed of SU-8. The thickness of the insulating layer 460 may be selected as large as possible to prevent the liquid crystal molecules 418 being affected by the conductors 430. Vias 450 or other electrical connections may be used to electrically interconnect the conductors 430 with the connectors 440 within the insulating layer 460.

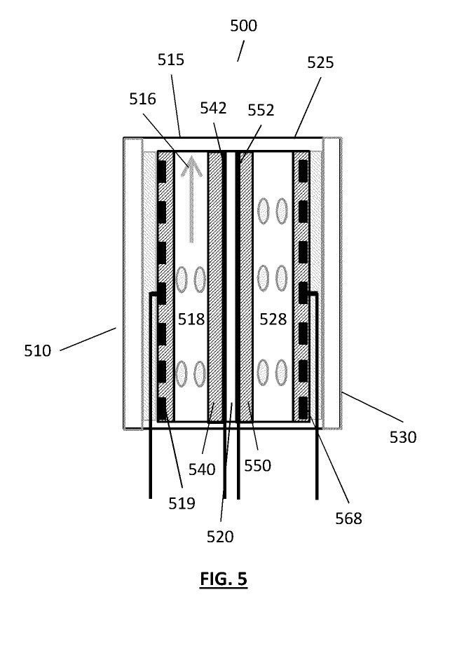

FIG. 5 is a schematic side view of a double cell LCAL according to one embodiment of the present invention. The double-cell LCAL 500 comprises a first reference plate 510, a ground plate 520 wherein the ground plate 520 is connected to the first reference plate 510 by a first connecting member 515. A first liquid crystal layer 518 is disposed between the first reference plate 510 and the ground plate 520, wherein the first liquid crystal layer 518 is aligned in a first direction 516. A first plurality of closed-loop electrodes 519 is disposed on the first reference plate 510, wherein the first plurality of electrodes 519 is configured to receive a first plurality of variable control voltages. A first uniform conducting layer 540 is disposed on a first surface 542 of the ground plate 520; while a second conducting layer 550 is disposed on a second surface 552 of the same ground plate 520. A second reference plate 530 is connected to the ground plate 520 by a second connecting member 525. A second liquid crystal layer 528 is disposed between the ground plate 520 and the second reference plate 530, wherein the second liquid crystal layer 528 is aligned at a second direction (not shown) perpendicular to the first direction 516 of the first liquid crystal layer 518, and a second set of closed-loop electrodes 568 is disposed on the second reference plate 530, wherein the second plurality of closed-loop electrodes 568 is configured to receive a second plurality of variable control voltages. The first surface 540 and the second surface 550 of the ground plate 520 need to be rubbed in the orthogonal direction. The fabrication process of the first reference plate 510 and the second reference plate 530 is the same as that of the reference plate of the single cell LCAL.

In endoscopy application, the light intensity is an important consideration. The double cell LCAL has the advantages of 100% light transmission. If the light polarized in “y” direction is focused by the first liquid crystal layer 518, then the light polarized in “x” direction is focused by the second liquid crystal layer 528 because the two layers of liquid crystal molecules are aligned perpendicular to each other. The double cell LCAL does not require the use of a polarizer. However, the distance from the first liquid crystal layer 518 to the second liquid crystal layer may introduce aberration. A super thin transparent substrate may be used as the shared ground plate to reduce this aberration. For example, a thin silicon dioxide layer of 0.1 mm, 0.2 mm, and 0.5 mm may be used as the ground plate. The aberration resulted from this small distance is minimal. Furthermore, the set of control voltages for each liquid crystal layer is adjustable independently. The aberration may be further reduced by the aberration correction subsystem.

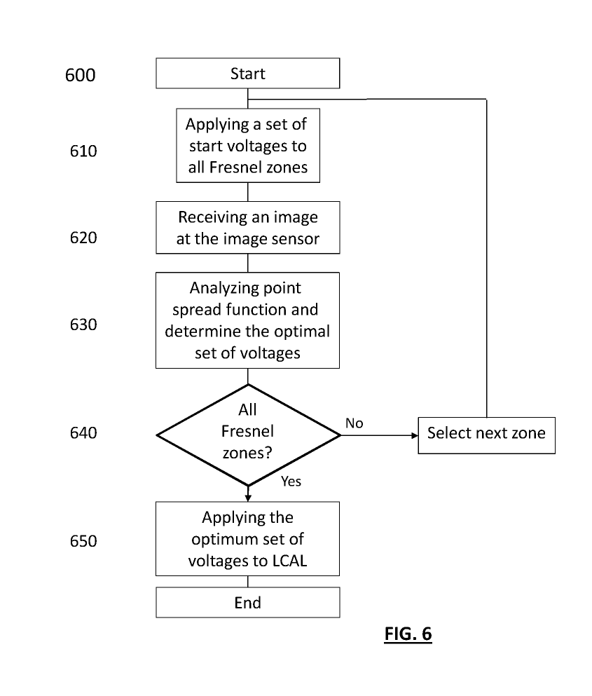

FIG. 6 is a flow diagram of the auto-focusing subsystem of the control system according to one embodiment of the present invention. In operation, a start voltage is applied to the two addressable conductors in one Fresnel zone. See block 610. The image of the internal structure formed by the optical system of the LCAL is received by the image sensor, as shown in block 620. The point spread function of the image is analyzed. As known to those skilled in the art, the point spread function (PSF) of an optical imaging system will represent the light distribution of a point after passing through the optical system. The set of optimum control voltages can be determined by analyzing the PSF distributions. Next, the voltage is increased from the start voltage to the end voltage by an incremental voltage, the image is received by the image sensor and the PSF is calculated and compared to that of the previous image. The optimum control voltage for each Fresnel zone can be determined, as shown in block 630. The next Fresnel zone is then selected, and the process is repeated. See blocks 640. After the PSFs have been analyzed for all the Fresnel zones, the final set of optimum control voltages are applied to the LCAL, as shown in block 650.

When the start voltage is very close to the optimum voltage, the auto-focusing process may be completed very fast, as short as few milliseconds; when the start voltage is far from the optimum voltage, the auto-focusing process may take much longer. A distance-sensing subsystem may be adapted to facilitate the process.

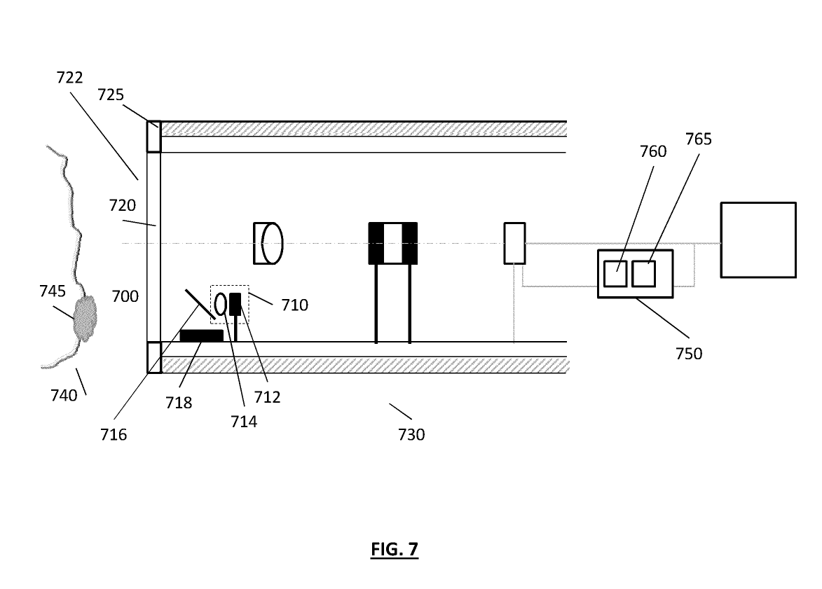

FIG. 7 is a schematic view of an endoscope comprises a distance sensing system according to one embodiment of the present invention. The endoscope comprises a distance-sensing system 700 located near the transparent window 720 at the distal end 722. The distance sensing system 700 comprises an LED assembly 710. The LED assembly comprises an LED 712, optically coupled to a collimating lens 714 such that the LED assembly 710 delivers collimated light. The distance sensing assembly 700 further comprises a beam splitter 716, which is used to direct a portion of the reflected light from a spot 745 of an observed internal structure 740 to a photodiode 718 adapted within the endoscope tube 730. The photodiode 718 receives the reflected light from the illumination of the LED 712, transfers the optical signal to the electrical signal, and sends the electrical signal to the control system 750. The control system 750 calculates the distance of the internal structure 740 to the LED 712. In some embodiments, the LED 712 is a single wavelength LED or infrared LED such that the wavelength of the LED 712 is different than that of the illumination system, thus the reflected light does not include the light from the illumination system 725. The control system 750 further comprises a look-up table 760. The look-up table 760 maps the set of desired control voltages to each Fresnel Zones with the calculated distance. The distance-sensing subsystem 700 can be adapted in conjunction with the auto-focusing subsystem 765. The set of desired control voltages from look-up table may be applied to the LCAL as the start voltage. The auto-focusing subsystem 765 may fine tune the set of control voltages and find the set of optimum voltages in milliseconds or tens of milliseconds.

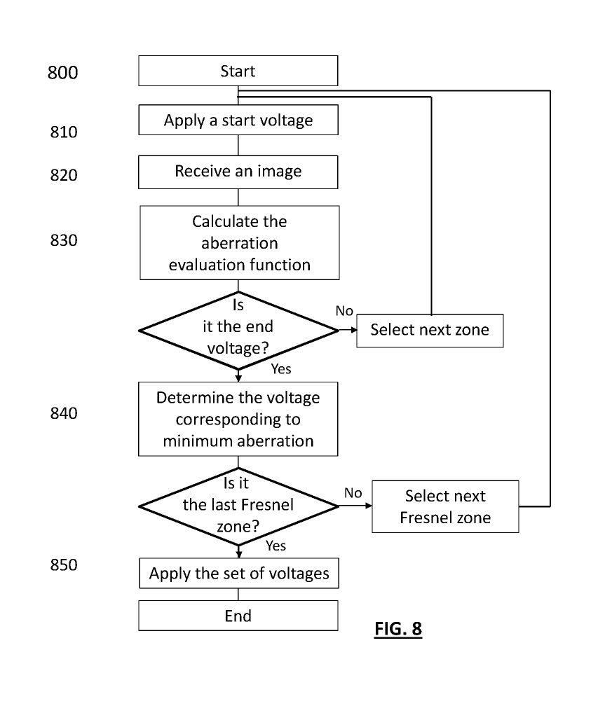

FIG. 8 is a flow diagram of an aberration correction subsystem of the endoscope control system according to one embodiment of the present invention. There are generally two sets of phase aberrations, static phase aberration and dynamic phase aberration. Dynamic phase aberration results from inaccurate applied voltages. Static aberration results from the optical system. In conventional glass lenses, there are conventional static aberrations such as chromatic aberration, spherical aberration, astigmatism, tilt, and field curvature, etc. The static phase aberration in LCAL includes quantization aberration and meshing aberration in addition to the conventional static aberrations. In some embodiments, the control system of an endoscope further comprises an aberration correction subsystem.

In order to minimize both dynamic and static aberrations, the aberration correction subsystem first applies a start voltage as shown in block 810. An image is received as shown in block 820. Then the aberration is evaluated by calculating an aberration evaluation function, as shown in block 830. The aberration evaluation function accounts for conventional aberrations including spherical aberration, astigmatism, tilt, field curvature and etc. Several methods may be used to design the aberration evaluation function. In one embodiment, the aberration evaluation function is designed using the “knife edging technique”. The image from an aberration free optical system has a sharp edge. Various kinds of aberration result in blurry images and fuzzy edges in the image with various characteristics. The aberration system analyzes the information related to the edges of the received image and calculates the aberration evaluation function. The information related to the edges including but not limited to the information such as the light intensity changing rate at all the edges of the image and the differences of the light intensity changing rate in different directions. The higher the sum of the light intensity changing rate at all the edges is, the smaller the aberration is the lower the differences of the light intensity changing rate in different directions are, the smaller the aberration is. For example, the aberration evaluation function may be defined as to be inversely proportional to the sum of the light intensity changing rate at all the edges and proportional to the difference of the light intensity changing rate in different directions in a simple model. The simple model is for illustrative purpose only, more sophisticated model may be developed within the scope of the invention. Next, the control voltage is increased in an incremental voltage, the aberration correction subsystem calculates the aberration evaluation function for the increased voltage of the Fresnel zone. After the control voltage reaches the end control voltage, the subsystem determines the control voltage corresponding to the minimum aberration evaluation function, see block 840. Then the process is repeated for each Fresnel zone. Lastly, the subsystem applies the set of control voltages corresponding to the minimum aberration evaluation function to the plurality of the closed-loop electrodes for all the Fresnel zones, as shown in block 850.

The control system for the double cell LCAL may also comprise the distance-sensing subsystem, the auto-focusing subsystem and the aberration correction system. The look-up table of the double cell LCAL maps the distance to the two sets of control voltages for both the first liquid crystal layer and the second liquid crystal layer. The auto-focusing subsystem for the double cell LCAL determines the two sets of optimum control voltages for both liquid crystal layers. The aberration correction subsystem determines the two sets of voltages corresponding to the minimum aberration for both liquid crystal layers.

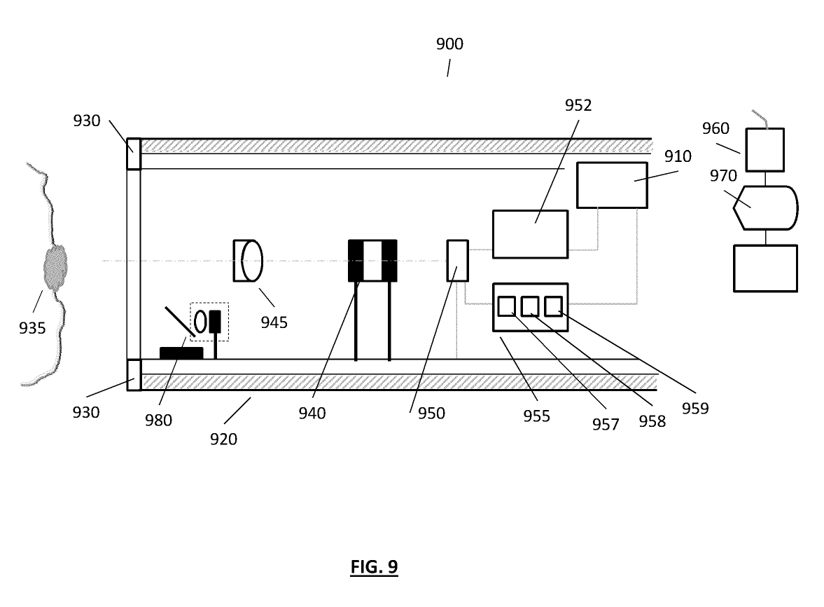

FIG. 9 is a schematic illustration of a wireless module of an endoscope with an electrically variable focal length according to one embodiment of the invention. The endoscope 900 is configured to use wireless communication. A battery 910 is located at the distal end of the elongate tube 920. The illumination system 930 and LCAL 940 are powered by the battery 910. The reflected light from the internal structure 935 passes through the objective lens 945 and the LCAL 940 and forms an image on the image sensor 950. Wireless transmitter 952 is connected to the image sensor 950 to receive image signals. It is also connected to the control system 955, wherein the control system is configured to electrically adjust the focal length. The wireless transmitter 952 further broadcasts the signals to the wireless transceiver 960 at the base, wherein the wireless transceiver 960 sends signals to the monitor 970. The control system 955 is connected to the image sensor 950. The wireless module may also comprise a distance-sensing system 980. The control system 955 comprises a look-up table 957, an auto-focusing subsystem 958 and an aberration correction system 959.

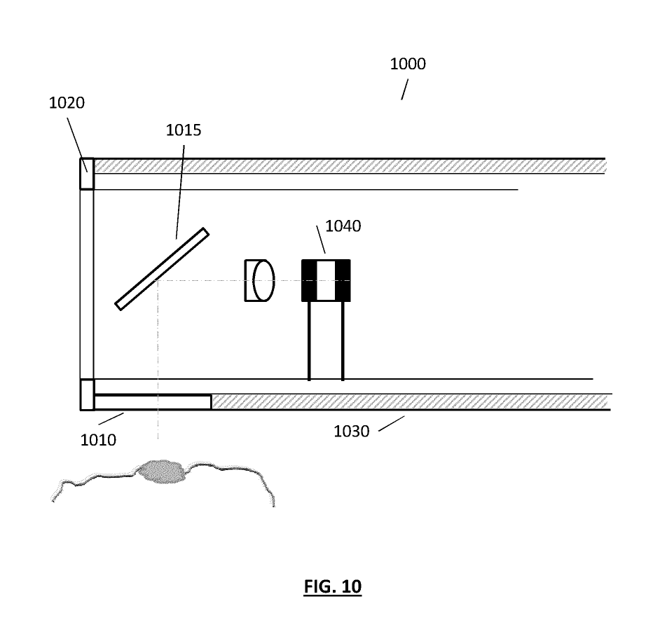

FIG. 10 is a schematic view of an endoscope with a transparent window at the side wall according to one embodiment of the present invention. The transparent window may have different configuration to accomplish the different viewing requirements. For clinical inspection and diagnosis, physicians need to observe the internal structures on the side walls. In one embodiment, a transparent window 1010 is mounted on the side wall 1030 at the distal end 1020. It will allow a physician to observe the side walls of the intestine, veins, and artery, etc. A mirror 1015 is used to redirect the light to pass through the LCAL 1040.

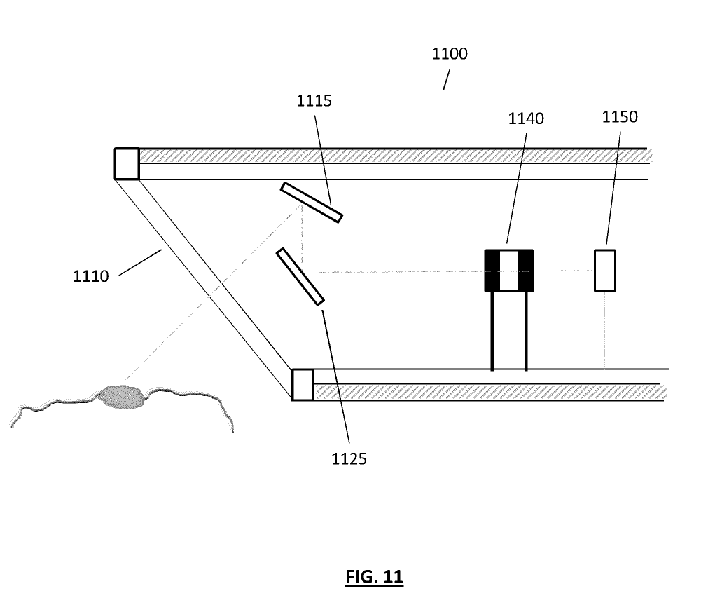

FIG. 11 is a schematic view of an endoscope with a front surface at an angle according to one embodiment of the present invention. In some clinical applications, the physicians need to observe both the front view and the side view. A transparent window 1110 is adapted to be at an angle such that both the front view and the side view are captured by the image sensor 1150. Two mirrors 1115 and 1125 are aligned to direct the light going through the LCAL 1140.

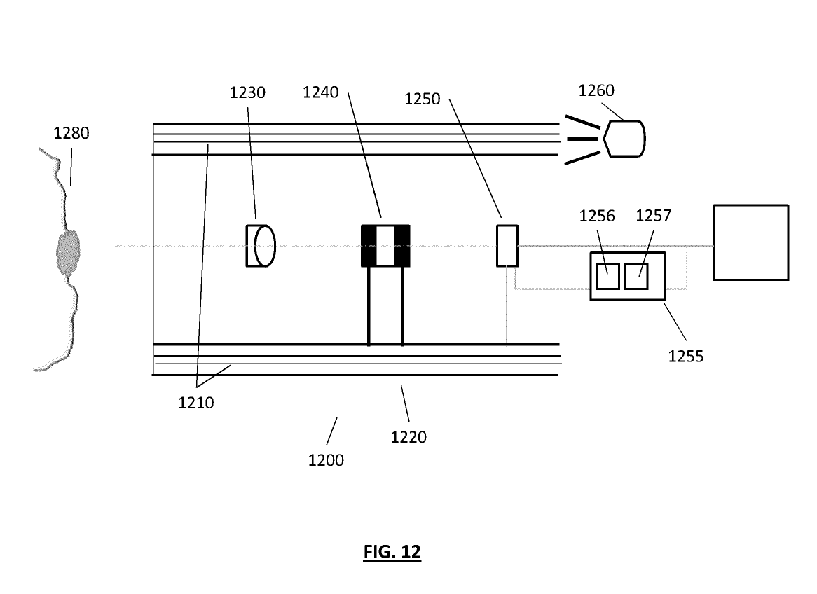

FIG. 12 is a schematic view of an endoscope with fiber bundles as illumination sources according to one embodiment of the present invention. Fiber bundles 1210 are located at the outer portion of the elongate tube 1220. Light from a light source 1260 passes through the fiber bundle 1210 and incidents on an internal structure 1280. The reflected light passes the objective lens 1230, the LCAL 1240 and forms an image on the image sensor 1250. The control system 1255 electrically adjusts the set of control voltages using the auto-focusing subsystem 1256 and the aberration correction subsystem 1257.

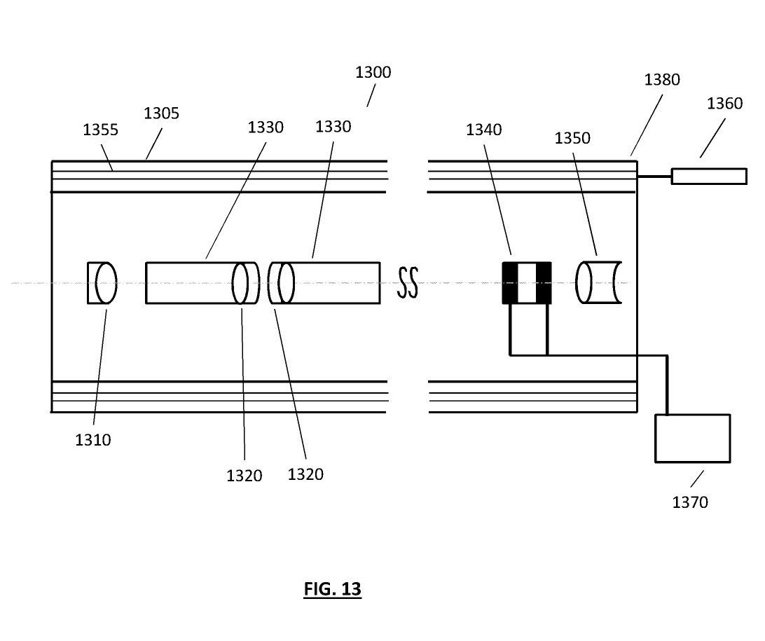

FIG. 13 is a schematic view of an endoscope with an eyepiece according to one embodiment of the present invention. In some circumstances, the physicians may prefer to observe the internal structure by their eyes. The optical imaging system of the endoscope 1300 comprises an objective lens 1310, relay lenses 1320, rod lenses 1330, an eye piece 1350 and an LCAL 1340. The light source 1360 delivers light to the internal structure through fiber bundles 1355 located at the outer portion of the elongate tube 1305. The light passes through the objective lens 1310, transmits by relay lenses 1320 and rod lenses 1330, and forms a virtual image through the eyepiece 1350 and LCAL 1340. The LCAL 1340 is located near the eyepiece 1350 at the proximal end 1380. The LCAL 1340 is configured to receive a set of control voltages by the control system 1370. The control system comprises a series of sets of control voltages. The physician may change the set of control voltages by manually changing the input to the control system, such as pushing a control button or selecting an input value from the menu on the control system. This configuration avoids introducing electricity into the human body.

The endoscope using an LCAL is capable of displaying a three-dimensional image because of fast response time of liquid crystal molecules to electrical signals. In one embodiment, the control system varies the set of control voltages such that the focal length of the LCAL is changed at an incremental step at a fast speed, for example, in a few kHz. Thus a series of two-dimensional images of an internal structure is received by the image sensor and sent to the control system. The control system further comprises an image processing subsystem which processes the series of two-dimensional images. As known to those skilled in the art, depth information may be extracted from each two-dimensional image based on the known focal length of the LCAL. The image processing subsystem then generates a corresponding in-focus depth-wise image. The imaging processing subsystem further generates a three-dimensional image from the set of in-focus depth-wise images taken at different focal lengths. When the imaging rate is fast enough, a three-dimensional image generated by imaging processing may be displayed by a conventional display device.

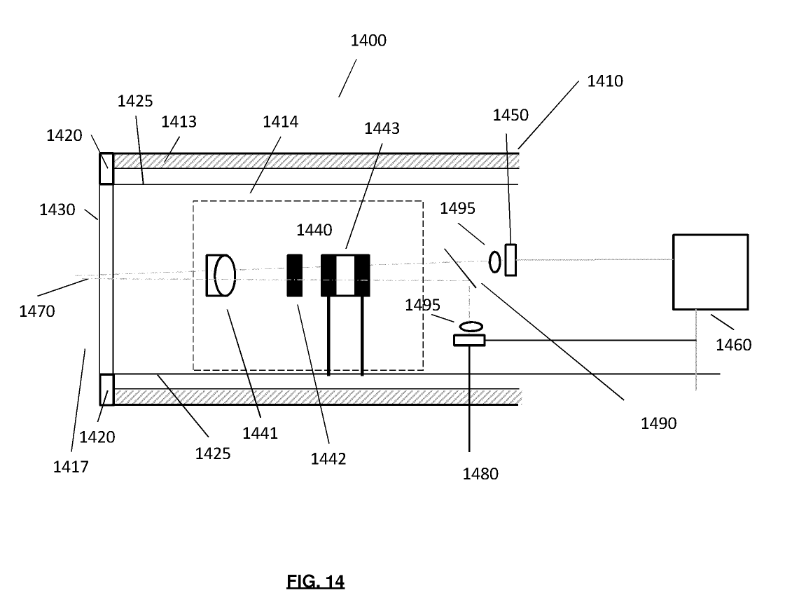

FIG. 14 is a schematic view of an endoscope capable of forming a three-dimensional image with an electrically adjustable focal length according to another embodiment of the present invention. The endoscope with an LCAL further comprises a second image sensor 1480 and a beam splitter 1490. The beam splitter is used to split the optical beam into two paths, wherein the optical axis of the second image sensor and the optical axis of the first image sensor 1450 have a small convergent angle. The small convergent angle of the two optical axis result in a three-dimensional image. In some embodiments, one or more relay lenses (not shown) may be used in the optical imaging system to form images into the image sensors. In some other embodiments, the image sensors may also be used with their focusing lenses 1495.

The endoscope need to be sterilized in an autoclave after observing a patient. In the autoclave sterilization process, the endoscope is exposed to a high pressure/high temperature water vapor at about 140° C. for about an hour. The endoscope is airtight and hermetically sealed to prevent the vapor penetrating into the inner portion of the tube. The components of the endoscope are required to be rigidly formed and be able to survive the high temperature. The LCAL is suitable for endoscope application because the liquid crystal materials can be designed to have a high operating temperature such as 400° C. Thus the LCAL is capable of withstanding repeated sterilization in an autoclave at 140° C. for about an hour.

While the present invention has been disclosed in example embodiments, those of ordinary skill in the art will recognize and appreciate that many additions, deletions and modifications to the disclosed embodiment and its variations may be implemented without departing from the scope of the invention.

What is claimed is:

1. An endoscope capable of electrically varying a focal length comprising:
an elongate tube;
a light source;
a window at a distal end of the elongate tube;
an optical imaging system within an inner portion of the elongate tube, comprising a liquid crystal adaptive lens (LCAL) comprising
a ground plate,
a first reference plate connected to the ground plate by a first connecting member,
a first liquid crystal layer disposed between the ground plate and the first reference plate, and
a first plurality of closed-loop electrodes disposed on the first reference plate in a concentric circular pattern, configured to receive a first plurality of variable control voltages;
a control system configured to adjust the first plurality of variable control voltages; and
a viewing system configured to receive an image from the optical imaging system.

2. The endoscope in claim 1, wherein the LCAL further comprises
a second reference plate wherein the second reference plate is connected to the ground plate by a second connecting member;
a second liquid crystal layer disposed between the second reference plate and the ground plate; and
a second plurality of closed-loop electrodes disposed on the second reference plate, configured to receive a second plurality of variable control voltages.

3. The endoscope in claim 1, wherein the first plurality of closed-loop electrodes comprise at least one subset of closed-loop electrodes, wherein the LCAL is capable of emulating a Fresnel phase profile with each subset of closed-loop electrodes comprising a Fresnel zone.

4. The endoscope in claim 1, wherein the LCAL further comprises at least one pair of conductors connected to at least two closed-loop electrodes; at least one connector connecting at least two closed-loop electrodes and each conductor of a respective pair of conductors.

5. The endoscope in claim 1, wherein the first plurality of closed-loop electrodes has a width of 10 nm and above, and a spacing of 10 nm and above.

6. The endoscope in claim 1, wherein the light source comprises solid state emitters.

7. The endoscope in claim 1, wherein the optical imaging system further comprises a fixed objective lens, aligned with an optical axis of the LCAL.

8. The endoscope in claim 1, wherein the view system comprises an image sensor and a display subsystem.

9. The endoscope in claim 1, wherein the view system comprises an eyepiece.

10. The endoscope in claim 1, wherein the control system further comprises an auto-focusing subsystem configured to calculate a point spread function for the received image and adjust the first plurality of variable control voltages by optimizing the point spread function.

11. The endoscope in claim 1, wherein the control system further comprises an aberration correction subsystem configured to calculate an aberration evaluation function for the received image and adjust the first plurality of variable control voltages to minimize the aberration evaluation function.

12. The endoscope in claim 1, wherein the endoscope further comprises a distance-sensing subsystem comprising an LED, a collimating lens, a beam splitter, a photodiode and a look-up table mapping a distance with the first plurality of variable control voltages.

13. The endoscope in claim 1, wherein the endoscope further comprises a wireless transmitter, a wireless transceiver and a battery within the elongate tube.

14. The endoscope in claim 1, wherein the window is adapted at a front surface at the distal end of the elongate tube.

15. The endoscope in claim 1, wherein the window is adapted at an angle to a front surface of the elongate tube, wherein the optical imaging system further comprises a first mirror and a second mirror to direct light along the direction of an optical axis of the LCAL.

16. The endoscope in claim 1, wherein the window is adapted to be at a side wall of the elongate tube, wherein the optical imaging system further comprises a mirror to direct light along the direction of an optical axis of the LCAL.

17. The endoscope in claim 1, wherein the endoscope is suitable to withstand the sterilization procedure in an autoclave at about 140° C.

18. An endoscope capable of electrically varying a focal length comprising:
an elongate tube;
an illumination system;
a transparent window at a distal end of the elongate tube;
an optical imaging system within an inner portion of the elongate tube, wherein the optical system comprises a liquid crystal adaptive lens (LCAL) comprising
a ground plate,
a first reference plate connected to the ground plate by a first connecting member,
a first liquid crystal layer disposed between the ground plate and the first reference plate,
a first plurality of closed-loop electrodes disposed on the first reference plate in a concentric circular pattern, configured to receive a first plurality of variable control voltages,
a second reference plate wherein the second reference plate is connected to the ground plate by a second connecting member,
a second liquid crystal layer disposed between the second reference plate and the ground plate, and
a second plurality of closed-loop electrodes disposed on the second reference plate, configured to receive a second plurality of variable control voltages;
a viewing system configured to receive an image from the optical system; and
a control system configured to adjust the first and the second plurality of variable control voltages.

19. A method of electrically varying a focal length of an endoscope comprising:
providing an elongate tube;
delivering a light through an illumination system;
providing an optical imaging system within an inner portion of the tube, wherein the optical imaging system comprises an LCAL comprising a ground plate, a first reference plate, a first liquid crystal layer, and a first plurality of closed-loop electrodes disposed on the first reference plate, configured to receive a first plurality of variable control voltages;
adjusting the first plurality of variable control voltages by a control system; and
receiving an image by a viewing system.

20. The method of electrically varying a focal length of an endoscope in claim 19, wherein the LCAL further comprises a second reference plate, a second liquid crystal layer, and a second plurality of closed-loop electrodes disposed on the second reference plate, configured to receive a second plurality of variable control voltages; wherein the control system further adjusts the second plurality of variable control voltages.
